(19) United States
(12) Patent Application Publication
(10) Pub. No.: US 2003/0224402 A1
(43) Pub. Date: Dec. 4, 2003

(54) Progression suppressed gene 13 (PSGen 13) and uses thereof

(76) Inventors: Paul B. Fisher, Scarsdale, NY (US); Dong-Chul Kang, Rutherford, NJ (US); Zao-Zhong Su, New York, NY (US)

Correspondence Address: BAKER & BOTTS, 30 ROCKEFELLER PLAZA, NEW YORK, NY, 10112

(21) Appl. No.: 10/373,556

(22) Filed: Feb. 24, 2003

Related U.S. Application Data
(63) Continuation of application No. PCT/US01/26795, filed on Aug. 27, 2001.
Application No. PCT/US01/26795 is a continuation-in-part of application No. 09/648,310, filed on Aug. 25, 2000.

(51) Int. Cl. C12Q 1/68; G01N 33/574; C07H 21/04; C12N 9/99; C12P 21/02; C12N 5/06

(52) U.S. Cl. 435/6; 435/7.23; 435/69.2; 435/184; 435/320.1; 435/325; 536/23.2

(57) ABSTRACT

The invention provides for isolated nucleic acids encoding Progression Suppressed Gene 13 (PS Gen 13) proteins, vectors comprising said nucleic acids, isolated PSGen 13 proteins, and methods of using such molecules to prevent the growth of cancer cells and/or new blood vessels and accordingly to treat patients suffering from cancer. It is based, at least in part, on the characterization of the complete cDNAs encoding rat and human PSGen 13, and on the discovery that increased levels of PSGen 13 expression can suppress the transformed phenotype and inhibit the activities of promoter elements associated with cancer progression and angiogenesis. In various embodiments, the present invention provides a method for inhibiting growth of a cancer cell which comprises contacting the cancer cell with a nucleic acid encoding a PSGen 13 protein, a PSGen 13 protein or a PSGen 13 activator compound in a sufficient amount so as to inhibit growth of the cancer cell. The invention also provides a method for treating cancer in a subject which comprises contacting a cell of the subject with a nucleic acid encoding a PSGen 13 protein in a sufficient amount so as to cause the cell to express the PSGen 13 protein, thereby treating cancer in the subject.

The invention disclosed herein was made with government support under National Institutes of Health Grant No. CA35675 from the U.S. Department of Health and Human Services. Accordingly, the U.S. government has certain rights herein.

This is a continuation-in-part of U.S. Ser. No. 09/648,310, the contents of which are hereby incorporated by reference in its entirety.

### 1. INTRODUCTION

The present invention relates to a gene which is expressed at lower levels in cells as they progress toward a malignant phenotype, and hence is referred to as Progression Suppressed Gene 13 (“PSGen 13”). The invention is based, at least in part, on the discovery of this novel gene and its encoded protein, and on the discovery that introducing PSGen13 into a malignant cell inhibits cancer cell growth and inhibits expression control elements of genes associated with malignancy and blood vessel formation.

### 2. BACKGROUND OF THE INVENTION

Changes in gene expression are important determinants of normal cellular physiology, including cell cycle regulation, differentiation, and development, and they directly contribute to abnormal cellular physiology, including developmental anomalies, aberrant programs of differentiation, and cancer (Watson and Margulies, 1993, Dev. Neurosci. 15:77-86; Winkles, 1998, Prog. Nucleic Acid Res. Mol. Biol. 58: 41-78; Fisher, ed., 1990, Model Cell Culture Systems for Studying Differentiation: Mechanisms of Differentiation, CRC Press, Boca Raton, Fla., Vol. 1; Fisher, ed., 1990, Modulation of Differentiation by Exogenous Agents: Mechanisms of Differentiation, CRC Press, Boca Raton, Fla., Vol. 2). In these contexts, the identification, cloning, and characterization of differentially expressed genes should provide relevant and important insights into the molecular determinants of processes such as growth, development, aging, differentiation, and cancer.

A number of procedures can be used to identify and clone differentially expressed genes. These include subtractive hybridization (Jiang and Fisher, 1993, Mol. Cell. Differ. 1: 285-299; Su and Fisher, 1997, Proc. Natl. Acad. Sci. U.S.A. 94: 9125-9130; Sagerström, et al., 1997, Annu. Rev. Biochem. 66: 751-783; Jiang et al., Oncogene 10: 1855-1864; Jiang, et al., 1995, Oncogene 11: 2477-2486; Jiang et al., 1996, Proc. Natl. Acad. Sci. U.S.A. 93: 9160-9165), differential RNA display (DDRT-PCR) (Watson and Margulies, 1993, Dev. Neurosci. 15: 77-86; Winkles,1998, Prog. Nucleic Acid Res. Mol. Biol. 58: 41-78; Liang and Pardee, 1992, Science 257: 967-971; Shen et al., 1995, Proc. Natl. Acad. Sci. U.S.A. 92: 6778-6782), RNA fingerprinting by arbitrarily primed PCR (McClelland et al., 1995, Trends Genet. 11: 242-246; Ralph et al., 1993, Proc. Natl. Acad. Sci. U.S.A. 90: 10710-10714), representational difference analysis (Hubank and Schatz, 1994, Nucleic Acids Res. 22: 5640-5648), serial analysis of gene expression (Velculescu, et al., 1995, Science 270: 484-487; Zhang, et al., 1997, Science 276: 1268-1272), electronic subtraction (Wan, et al., 1996, Nat. Biotechnol. 14: 1685-1691; Adams et al., 1993, Nat. Genet. 4: 256-267), and combinatorial gene matrix analysis (Schena et al., 1995, Science 270: 467-470).

The DDRT-PCR approach developed by Liang and Pardee (1992, Science 257: 967-971) has gained wide popularity in analyzing and cloning differentially expressed genes. DDRT-PCR is a powerful methodology in which a vast number of mRNA species (>20,000, if no redundancy occurs) can be analyzed with only a small quantity of RNA (approx. 5 μg) (Id.). DDRT-PCR is often the method of choice when the RNA source is limiting, such as tissue biopsies. A direct advantage of DDRT-PCR is the ability to identify and isolate both up- and down-regulated differentially expressed genes in the same reaction. Furthermore, the DDRT-PCR technique permits the display of multiple samples in the same gel, which is useful in defining specific diagnostic alterations in RNA species and for temporally analyzing gene expression changes.

However, the DDRT-PCR technique is not problem-free (Debouck, 1995, Curr. Opin. Biotechnol. 6: 597-599). Difficulties encountered when using standard DDRT-PCR include a high incidence of false positives and redundant gene identification, poor reproducibility, biased gene display, and lack of functional information about the cloned cDNA. Furthermore, poor separation can mask differentially expressed genes of low abundance under the intense signals generated by highly expressed genes. The generation of false positives and redundancy can be highly problematic, resulting in an inordinate expenditure of resources to confirm appropriate differential expression and uniqueness of the isolated cDNAs. The cDNAs must be isolated from the gels in pure form (contamination of bands with multiple sequences complicates clone identification), reamplified, placed in an appropriate cloning vector, analyzed for authentic differential expression, and, finally, sequenced. These limitations of the standard DDRT-PCR approaches emphasize the need for improvements in this procedure to more efficiently and selectively identify differentially expressed genes.

Subtractive hybridization, in which hybridization between tester and driver is followed by selective removal of common gene products, enriches for unique gene products in the tester cDNA population and reduces the abundance of common cDNAs (Sagerström, et al., 1997, Alinu. Rev. Biochem. 66: 751-783). A subtracted cDNA library can be analyzed to identify and clone differentially expressed genes by randomly picking colonies or by differential screening (Rangnekar et al., 1992, J. Biol. Chem. 267: 6240-6248; Wong et al., 1997, Semin. Immunol. 9: 7-16; Maser and Calvet, 1995, Semin. Nephrol. 15: 29-42). Although subtractive hybridization has been used successfully to clone a number of differentially expressed genes (Jiang and Fisher, 1993, Mol. Cell. Differ. 1: 285-299; Su et al., 1997, Proc. Natl. Acad. Sci. U.S.A. 94: 9125-9130; Jiang et al., Oncogene 10: 1855-1864; Jiang, et al., 1995, Oncogene 11: 2477-2486), this approach is labor-intensive and does not result in isolation of the full spectrum of genes displaying altered expression (Sagerström, et al., 1997, Annu. Rev. Biochem. 66: 751-783; Wan et al., 1996, Nat. Biotechnol. 14: 1685-1691).

In principle, DDRT-PCR performed with subtracted RNA or cDNA samples should provide a powerful strategy to clone up- and down-regulated gene products. This approach should combine the benefits of both techniques, resulting in the enrichment of unique sequences and a reduction or elimination of common sequences. This scheme also should result in a consistent reduction in band complexity on a display gel, thereby permitting a clearer separation of cDNAs, resulting in fewer false positive reactions. Additionally, it should be possible to use fewer primer sets for reverse transcription and PCR reactions to analyze the complete spectrum of differentially expressed genes. Of particular importance for gene identification and isolation, rare gene products that are masked by strong common gene products should be displayed by using subtraction hybridization in combination with DDRT-PCR. In addition, the DDRT-PCR approach with subtractive libraries also could prove valuable for efficiently screening subtracted cDNA libraries for differentially expressed genes. However, even though subtraction hybridization plus DDRT-PCR appears attractive for the reasons indicated above, a previous attempt to use this approach has proven of only marginal success in consistently reducing the complexity of the signals generated, compared with the standard DDRT-PCR scheme (Hakvoort et al., 1994, Nucleic Acids Res. 22: 878-879).

Kang et al. (1998, Proc. Natl. Acad. Sci. U.S.A. 95:13788-13793) used a reciprocal subtraction differential RNA display (“RSDD”) approach that efficiently and consistently reduces the complexity of DDRT-PCR and resulted in the identification and cloning of genes displaying anticipated differential expression. The model used for RSDD was an adenovirus-transformed rat embryo cell line, E11, that acquires an aggressive oncogenic progression phenotype when injected into athymic nude mice and reestablished in cell culture (E11-NMT) (Su and Fisher, 1997, Proc. Natl. Acad. Sci. U.S.A. 94: 9125-9130; Reddy et al., 1993, in Chromosome and Genetic Analysis: Methods in Molecular Genetics, ed. Adolph, K. W. (Academic, Orlando, Fla.), Vol. 1, pp. 68-102; Babiss et al., 1985, Science 228: 1099-1101). Injection of E11 cells into nude mice results in tumors in 100% of animals with a tumor latency time of about 35-40 days whereas E11-NMT cells form tumors in 100% of nude mice with a tumor latency time of 15-20 days (Id.). Additionally, E11 cells form colonies in agar with an efficiency of approx. 3% whereas E11-NMT display an agar cloning efficiency of >30% (Id.). The increased tumorigenicity and enhanced anchorage independence phenotypes are key indicators of tumor progression in the E11/E11-NMT model system (Id.).

Kang et al., supra, reported that RSDD resulted in the identification of genes displaying elevated expression in progressed rat tumor cells [progression-elevated gene (“PEGen genes”)] and suppressed expression in progressed tumor cells [progression-suppressed gene (“PSGen genes”)]. One of the rat genes identified by Kang et al. is (rat) PSGen 13 (see also International Patent Application No. PCT/US99/04323 by inventors Fisher et al.), but the full-length cDNA had not been isolated and therefore neither its sequence nor the sequence of its encoded protein were reported. The present invention relates to the cloning and characterization of the complete rat and human PSGen 13 cDNAs, and provides their nucleic acid and encoded amino acid sequences, and moreover demonstrates the suppressive effects of increased levels of PSGen 13 on the malignant phenotype.

### 3. SUMMARY OF THE INVENTION

The invention provides for isolated nucleic acids encoding Progression Suppressed Gene 13 (PS Gen 13) proteins, vectors comprising said nucleic acids, isolated PSGen 13 proteins, and methods of using such molecules to prevent the growth of cancer cells and/or new blood vessels and accordingly to treat patients suffering from cancer. It is based, at least in part, on the cloning and characterization of the complete cDNAs encoding rat and human PSGen 13, and on the discovery that increased levels of PSGen 13 expression can suppress the transformed phenotype and inhibit the activities of promoter elements associated with cancer progression and angiogenesis. It is further based on the discovery that the chromosomal location of human PSGen 13 is a region which has been observed to contain deletions in patients suffering from a variety of cancers, consistent with its observed tumor suppressor activity.

In various embodiments, the present invention provides a method for inhibiting growth of a cancer cell which comprises contacting the cancer cell with a nucleic acid encoding a PSGen 13 protein, a PSGen 13 protein or a PSGen 13 activator compound in a sufficient amount so as to inhibit growth of the cancer cell. The invention also provides a method for treating cancer in a subject which comprises contacting a cell of the subject with a nucleic acid encoding a PSGen 13 protein in a sufficient amount so as to cause the cell to express the PSGen 13 protein, thereby treating cancer in the subject.

3.1. Definitions

A DNA “coding sequence” or a “nucleotide sequence encoding” a particular protein, is a DNA sequence which is transcribed and translated into a polypeptide in vivo or in vitro when placed under the control of appropriate regulatory sequences. The boundaries of the coding sequence are determined by a start codon at the 5′-(amino) terminus and a translation stop codon at the 3′-(carboxy) terminus. A coding sequence can include, but is not limited to, procaryotic sequences, cDNA from eucaryotic mRNA, genomic DNA sequences from eucaryotic (e.g., mammalian) sources, viral RNA or DNA, and even synthetic nucleotide sequences. A transcription termination sequence will usually be located 3′to the coding sequence.

The term DNA “control sequences” refers collectively to promoter sequences, polyadenylation signals, transcription termination sequences, upstream regulatory domains, enhancers, and the like, untranslated regions (“UTRs”), including 5′-UTRs and 3′-UTRs, which collectively provide for the transcription and translation of a coding sequence in a host cell.

A control sequence “directs the transcription” of a coding sequence in a cell when an RNA polymerase will bind the promoter sequence and transcribe the coding sequence into mRNA, which is then translated into the polypeptide encoded by the coding sequence.

As used herein “enhancer element” is a nucleotide sequence that increases the rate of transcription of a therapeutic gene or genes of interest but does not have promoter activity.

As used herein, the term “gene” refers to a nucleic acid directly or indirectly encoding a product. For example, cDNA encoding a PSGen 13 protein would be considered a “PSGen 13 gene”, as would genomic DNA from which a PSGen 13-encoding mRNA could be transcribed.

A “heterologous” region of a DNA construct is an identifiable segment of DNA within or attached to another DNA molecule that is not found in association with the other molecule in nature. An example of a heterologous coding sequence is a construct where the coding sequence itself (e.g., the coding sequence of PSGen 13 mutant or fragment of nucleic acid encoding PSGen 13) is not found in nature (e.g., synthetic sequences having codons different from the native gene). Likewise, a chimeric sequence, comprising a heterologous structural gene and a gene encoding a PSGen 13 or a portion of such gene, linked to a tissue specific promoter, whether derived from the same or a different gene, will be considered heterologous since such chimeric constructs are not normally found in nature. Allelic variation or naturally occurring mutational events do not give rise to a heterologous region of DNA, as used herein.

As used herein “nucleic acid molecule” includes both DNA and RNA and, unless otherwise specified, includes both double-stranded and single-stranded nucleic acids. Also included are hybrids such as DNA-RNA hybrids. Reference to a nucleic acid sequence can also include modified bases as long as the modification does not significantly interfere either with binding of a ligand such as a protein by the nucleic acid or Watson-Crick base pairing, unless such interference is intended or desirable.

“Operably linked” refers to an arrangement of nucleotide sequence elements wherein the components so described are configured so as to perform their usual function. Thus, control sequences operably linked to a coding sequence are capable of effecting the expressing of the coding sequence. The control sequences need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

Two DNA or polypeptide sequences are “substantially homologous” when at least about 80% (preferably at least about 90%, and most preferably at least about 95%) of the nucleotides or amino acids match over a defined length of the molecule. As used herein, “substantially homologous” also refers to sequences showing at least about 80%, preferably at least about 90% and more preferably at least about 95% identity to the specified DNA or polypeptide sequence. DNA sequences that are substantially homologous can be identified in a Southern hybridization experiment under, for example, stringent conditions, as defined for the particular system. Defining appropriate hybridization conditions is within the skill of the art. See, e.g., Ausubel et al., 1989, Current Protocols in Molecular Biology, Vol. I, Green Publishing Associates, Inc., and John Wiley & Sons, Inc. New York.

As used herein “therapeutic gene” means DNA encoding an amino acid sequence corresponding to a functional protein capable of exerting a therapeutic (e.g., growth inhibitory, differentiating, and/or apoptosis inducing) effect on cancer cells or having a regulatory effect on the expression of a function in cells.

A cell has been “transformed” by exogenous DNA when such exogenous DNA has been introduced inside the cell membrane or, in the case of bacteria, the cell wall. Exogenous DNA may or may not be integrated (covalently linked) into chromosomal DNA making up the genome of the cell. In procaryotes and yeasts, for example, the exogenous DNA may be maintained on an episomal element, such as a plasmid. In eucaryotic cells, a stably transformed cell is generally one in which the exogenous DNA has become integrated into the chromosome so that it is inherited by daughter cells through chromosome replication, or one which includes stably maintained extrachromosomal plasmids. This stability is demonstrated by the ability of the eucaryotic cell to establish cell lines or clones comprised of a population of daughter cells containing the exogenous DNA.

### 4. BRIEF DESCRIPTION OF THE FIGURES

FIG. 1. Nucleotide and predicted amino acid sequence of the rat PSGen 13 gene (designated rPSGen 13), SEQ ID NOS: 1 and 2, respectively. Starting ATG of the open reading frame and the stop codon are circled and the poly(A) signal is underlined.

FIG. 2. Nucleotide and predicted amino acid sequence of the human PSGen 13 gene (designated HuPSGen 13), SEQ ID NOS: 3 and 4, respectively. The starting ATG of the open reading frame and stop codon are circled and the poly(A) signal is underlined.

FIG. 3. Nucleotide sequence comparison between the rat PSGen 13; SEQ ID NO: 1) and human (HuPSGen 13; SEQ ID NO: 3) cDNAs. The start and stop codons of the rat PSGen 13 and human HuPSGen 13 genes are in bold-face type.

FIG. 4. Amino acid sequence comparison between the rat PSGen 13 and human HuPSGen 13 proteins (SEQ ID NOS: 2 and 4, respectively). Conservative substitutions are indicated by a “:” and semi-conservative substitutions are indicated by a “.”.

FIG. 5. Differential expression of rPSGen 13 identified by RSDD and reverse Northern blotting in a large panel of rodent cells displaying differences in transformation progression. A Northern blot of cells displaying various stages of transformation progression was probed with a ³²P-radiolabeled rPSGen 13 cDNA initially identified by RSDD and reverse Northern blotting (Kang et al., 1998, Proc. Natl. Acad. Sci. U.S.A. 95:13788-13793). The RNA was obtained from cell types represented in the Northern blot in lanes, left to right, as follows: unprogressed E11 cells (“E11 (−)”); progressed E11-NMT (+) cells; CREF×E11-NMT F1 (−) unprogressed cells (where “Cell Type A×Cell Type B” designates a somatic cell hybrid between a Type A cell and a Type B cell); CREF×E11-NMT F2 (−) unprogressed cells; CREF×E11-NMT R1 (+) progressed cells; CREF×E11-NMT R2 (+) progressed cells; E11×E11-NMT A6 (−) unprogressed cells; E11 ×E11-NMT A6TD (+) nude mouse tumor-derived progressed cells; E11×E11-NMT 3b (−) unprogressed cells; E11×E11-NMT IIa (+) progressed cells; E11-NMT AZA B1 (−) unprogressed 5-azacytidine treated E11-NMT clone; E11-NMT AZA C1 (−) unprogressed 5-azacytidine treated E11-NMT clone; E11-Ras R12 progressed cells; and E11-HPV E6/E7, an E11 clone transformed by the E6 and E7 region of HPV-18. Equal loading of RNAs is demonstrated by ethidium bromide (EtBr) staining. Data is from Kang et al., 1998, Proc. Natl. Acad. Sci. U.S.A. 95:13788-13793.

FIG. 6. rPSGen 13 suppresses anchorage independent growth in E11-NMT cells. Agar cloning efficiencies of the indicated cell types were determined as described previously (Fisher et al., 1979, Cancer Res. 39:3051-3057; Fisher et al., 1979, Nature 281:591-594; Fisher et al., 1979, Cell 18:695-705). Cell types include, E11, E11-NMT and PSGen 13 transfected E11-NMT clones, designed as NMT-PSG13 cl 3, 5, 6, 7, 8, 9, 10, 11 and 12. Triplicate samples varied by <10% and replicate assays varied by <15%.

FIG. 7. Northern blotting analysis of rat PSGen 13 and GAPDH expression in E11, E11-NMT and NMT-PSG13 clones. Fifteen micrograms of cellular RNA isolated from the indicated cell types were electrophoresed, transferred to nylon membranes and hybridized with a rat PSGen 13 cDNA and then stripped and probed with GAPDH as previously described (Su et al., 1997, Proc. Natl. Acad. Sci. U.S.A. 94:9125-9130; Su et al., 1999, Proc. Natl. Acad. Sci. U.S.A. 96:15115-15120).

FIG. 8. Rat PSGen 13 inhibits anchorage independent growth in DU-145 human prostate carcinoma cells. Agar cloning efficiencies of the indicated cell types were determined as described previously (Fisher et al., 1979, Cancer Res. 39:3051-3057; Fisher et al., 1979, Nature 281:591-594; Fisher et al., 1979, Cell 18:695-705). Cell types include, DU-145, DU-145 vector transformed clone (DU-145/Vec) and rat PSGen 13 transfected DU-145 clones, designed as DU-PSG13 cl 11, 12, 13, 14, 15 and 17. Triplicate samples varied by <10% and replicate assays varied by <12%.

FIG. 9. Full length PEG-3 promoter-luciferase activity in E11, E11-NMT and NMT-PSG13 clones. Different cell types were co-transfected with 5 μg of the full length PEG-Prom and 1 μg of a pSV-β-galactosidase plasmid and luciferase activity was determined as described in Materials and Methods 48 hr later. The results are standardized by β-galactosidase activity and represent the average of 3 independent experiments that varied by <15%.

FIG. 10. VEGF promoter-luciferase activity in E11, E11-NMT and NMT-PSG13 clones. Different cell types were co-transfected with 5 μg of the VEGF-Prom-luciferase (Su et al., 1999, Proc. Natl. Acad. Sci. U.S.A. 96:15115-15120) and 1 μg of a pSV-β-galactosidase plasmid and luciferase activity was determined as described in Materials and Methods 48 hr later. The results are standardized by β-galactosidase activity and represent the average of 3 independent experiments that varied by <15%.

FIG. 11. HuPSGen 13 selectively suppressed monolayer colony formation in Ha-ras transformed rat embryo fibroblast cells.

FIG. 12. HuPSGen 13 suppressed the transformed phenotype of MCF-7 human breast carcinoma cells, as demonstrated by a decrease in cloning efficiency in agar.

### 5. DETAILED DESCRIPTION OF THE INVENTION

For clarity of presentation, and not by way of limitation, the detailed description of the invention is divided into the following subsections:

(i) PSGen 13 nucleic acids;

(ii) PSGen 13 proteins;

(iii) antibodies to PSGen 13 proteins; and

(iv) uses of PSGen13.

5.1 PSGen 13 Nucleic Acids

The present invention provides for PSGen 13 nucleic acids, including PSGen genes and related molecules. The term “PSGen 13” refers to a PSGen 13 molecule without regard to species.

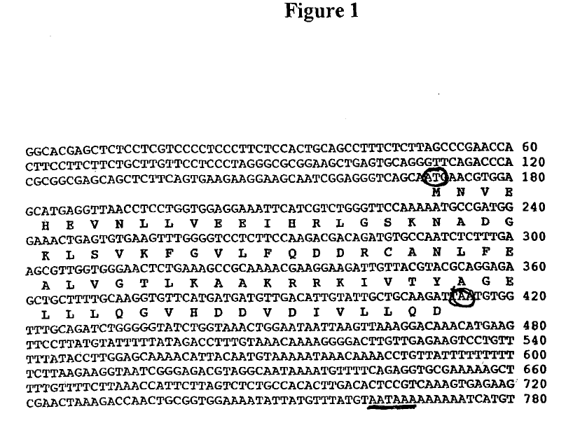

In particular non-limiting embodiments, the present invention provides for a rat PSGen 13 (“rPSGen 13”) gene having a sequence as set forth in FIG. 1 and SEQ ID NO: 1 or SEQ ID NO:5 and as deposited with the American Type Culture Collection (“ATCC”), an organization having an address at 10801 University Blvd., Manassas, Va. 20110-2209 and assigned Accession Number PTA-2414, and substantially homologous molecules. The deposit is a rPSGen 13 c DNA insert in a pcDNA3.1 (+) plasmid vector, within the EcoRI-Xho I cloning site. The insert is about 0.8 kb in length. The sense strand promoter of the plasmid is T7. The plasmid carries resistance genes to ampicillin and neomycin. The insert origin is EST clone ATCC #2005777 (see below). The rat tissue used to isolate the cDNA was adrenal gland tissue.

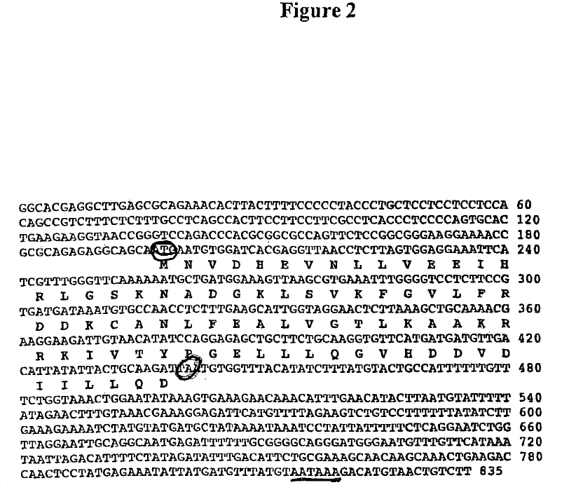

In other particular non-limiting embodiments, the present invention provides for a human PSGen 13 (“HuPSGen 13”) gene having a sequence as set forth in FIG. 2 and SEQ ID NO:3 or SEQ ID NO:6, and as deposited with the ATCC and assigned Accession Number PTA-2413, and substantially homologous molecules. The deposit is an insert within a plasmid vector, pT7T3-Pac. It is within the EcoRI-Not I cloning site. The insert is about 0.83 kb in length. The plasmid carries a resistance gene to ampicillin. The insert origin is EST clone ATCC #2525262 (see below). The human tissue used to isolate the DNA was human kidney tissue.

The present invention further provides for nucleic acids which would hybridize with the rPSGen 13 gene and/or the HuPSGen 13 gene under stringent hybridization conditions, such as e.g., hybridization in 0.5 M NaHPO₄, 7 percent sodium dodecyl sulfate (“SDS”), 1 mM ethylenediamine tetraacetic acid (“EDTA”) at 65° C., and washing in 0.1×SSC/0.1 percent SDS at 68° C. (Ausubel et al., 1989, Current Protocols in Molecular Biology, Vol. I, Green Publishing Associates, Inc., and John Wiley & Sons, Inc. New York, at p. 2.10.3). As such, the present invention encompasses PSGen 13 genes of other species, including a PSGen 13 gene of a non-human primate, a mouse, or a cow, as well as nucleic acid probes and antisense molecules (which could be used, for example, to support a transformed phenotype).

The present invention also provides for nucleic acids encoding the amino acid sequences set forth for the rPSGen 13 and HuPSGen 13 proteins in FIGS. 1 and 2, and as set forth in SEQ ID NOS: 2 and 4, and for nucleic acids that hybridize to such PSGen 13 protein-encoding nucleic acids under stringent conditions.

The nucleic acid sequences set forth in FIGS. 1 and 2 may be used to identify primer molecules which may be used to obtain a PSGen 13 gene by amplification using standard techniques.

The present invention provides for a PSGen 13 gene, such as the rPSGen 13 gene or the HuPSGen 13 gene, in an expressible form. An “expressible form” is one which contains the necessary elements and control sequences for transcription and/or translation. For example, a PSGen 13 gene may be operatively linked to a suitable promoter element (which may be a PSGen 13 promoter or a heterologous promoter), and may comprise an enhancer element, transcription initiation and termination sites, nucleic acid encoding a nuclear localization sequence, ribosome binding sites, polyadenylation sites, mRNA stabilizing sequences, etc.

Although expression in procaryotes is not precluded, in preferred embodiments the present invention provides for a PSGen 13 gene, such as the rPSGen 13 gene or the HuPSGen 13 gene, in a form expressible in eucaryotic cells. Accordingly, the PSGen 13 gene may be operatively linked, for example, to a promoter element active in eucaryotic cells. Suitable promoters may include the cytomegalovirus immediate early promoter, the Rous sarcoma virus long terminal repeat promoter, the human elongation factor 1α promoter, the human ubiquitin c promoter, etc. It may be desirable, in certain embodiments of the invention, to use an inducible promoter. Non-limiting examples of inducible promoters include the murine mammary tumor virus promoter (inducible with dexamethasone); commercially available tetracycline-responsive or ecdysone-inducible promoters, etc. In specific non-limiting embodiments of the invention, the promoter may be selectively active in cancer cells, such as the prostate specific antigen gene promoter (O'Keefe et al., 2000, Prostate 45:149-157), the kallikrein 2 gene promoter (Xie et al., 2001, Human Gene Ther. 12:549-561), the human alpha-fetoprotein gene promoter (Ido et al., 1995, Cancer Res. 55:3105-3109), the c-erbB-2 gene promoter (Takakuwa et al., 1997, Jpn. J. Cancer Res. 88:166-175), the human carcinoembryonic antigen gene promoter (Lan et al., 1996, Gastroenterol. 111:1241-1251), the gastrin-releasing peptide gene promoter (Inase et al., 2000, Int. J. Cancer 85:716-719). the human telomerase reverse transcriptase gene promoter (Pan and Koenman, 1999, Med. Hypotheses 53:130-135), the hexokinase II gene promoter (Katabi et al., 1999, Human Gene Ther. 10:155-164), the L-plastin gene promoter (Peng et al., 2001, Cancer Res. 61:4405-4413), the neuron-specific enolase gene promoter (Tanaka et al., 2001, Anticancer Res. 21:291-294), the midkine gene promoter (Adachi et al., 2000, Cancer Res. 60:4305-4310), the human mucin gene MUC1 promoter (Stackhouse et al., 1999, Cancer Gene Ther. 6:209-219), and the human mucin gene MUC4 promoter (Genbank Accession No. AF241535), which is particularly active in pancreatic cancer cells (Perrais et al., 2001, published on Jun. 19, 2001 by J Biol. Chem., “JBC Papers in Press” as Manuscript M104204200).

It should be noted that expressed sequence tag (“EST”) clones containing, unbeknownst to the initial depositors, rPSGen 13 and HuPSGen 13 amino acid sequence encoding sequences, were deposited with the ATCC prior to the filing date of U.S. Ser. No. 09/648,310 and had been assigned Accession Numbers 2005777 and 2525262, respectively. These clones were used in the characterization and cloning of the complete rPSGen 13 and HuPSGen 13 genes in the present invention (see Section 6, below). However, at the time the ESTs were deposited the complete genes were not known, and neither the identity nor the function of the protein encoded by the ESTs was known.

A PSGen 13 nucleic acid, such as a rPSGen 13 gene or a HuPSGen 13 gene operatively linked to a promoter element operative in a eucaryotic cell, may be comprised, e.g. together with other expression related elements, gene(s) associated with antibiotic resistance, etc., in a vector molecule. Examples of suitable vector molecules include but are not limited to include virus-based vectors and non-virus based DNA or RNA delivery systems. Examples of appropriate virus-based gene transfer vectors include, but are not limited to, those derived from retroviruses, for example Moloney murine leukemia-virus based vectors such as LX, LNSX, LNCX or LXSN (Miller and Rosman, 1989, Biotechniques 7:980-989); lentiviruses, for example human immunodeficiency virus (“HIV”), feline leukemia virus (“FIV”) or equine infectious anemia virus (“EIAV”)-based vectors (Case et al., 1999, Proc. Natl. Acad. Sci. U.S.A. 96: 22988-2993; Curran et al., 2000, Molecular Ther. 1:31-38; Olsen, 1998, Gene Ther. 5:1481-1487; U.S. Pat. Nos. 6,255,071 and 6,025,192); adenoviruses (Zhang, 1999, Cancer Gene Ther. 6(2):113-138; Connelly, 1999, Curr. Opin. Mol. Ther. 1(5):565-572; Stratford-Perricaudet, 1990, Human Gene Ther. 1:241-256; Rosenfeld, 1991, Science 252:431-434; Wang et al., 1991, Adv. Exp. Med. Biol. 309:61-66; Jaffe et al., 1992, Nat. Gen. 1:372-378; Quantin et al., 1992, Proc. Natl. Acad. Sci. U.S.A. 89:2581-2584; Rosenfeld et al., 1992, Cell 68:143-155; Mastrangeli et al., 1993, J. Clin. Invest. 91:225-234; Ragot et al., 1993, Nature 361:647-650; Hayaski et al., 1994, J. Biol. Chem. 269:23872-23875; Bett et al., 1994, Proc. Natl. Acad. Sci. U.S.A. 91:8802-8806), for example Ad5/CMV-based E1-deleted vectors (Li et al., 1993, Human Gene Ther. 4:403-409); adeno-associated viruses, for example pSub201-based AAV2-derived vectors (Walsh et al., 1992, Proc. Natl. Acad. Sci. U.S.A. 89:7257-7261); herpes simplex viruses, for example vectors based on HSV-1 (Geller and Freese, 1990, Proc. Natl. Acad. Sci. U.S.A. 87:1149-1153); baculoviruses, for example AcMNPV-based vectors (Boyce and Bucher, 1996, Proc. Natl. Acad. Sci. U.S.A. 93:2348-2352); SV40, for example SVluc (Strayer and Milano, 1996,Gene Ther. 3:581-587); Epstein-Barr viruses, for example EBV-based replicon vectors (Hambor et al., 1988, Proc. Natl. Acad. Sci. U.S.A. 85:4010-4014); alphaviruses, for example Semliki Forest virus- or Sindbis virus-based vectors (Polo et al., 1999, Proc. Natl. Acad. Sci. U.S.A. 96:4598-4603); vaccinia viruses, for example modified vaccinia virus (MVA)-based vectors (Sutter and Moss, 1992, Proc. Natl. Acad. Sci. U.S.A. 89:10847-10851) or any other class of viruses that can efficiently transduce human tumor cells and that can accommodate the nucleic acid sequences required for therapeutic efficacy. It may be desirable, for certain embodiments, to utilize a shuttle vector.

The present invention provides for compositions comprising an above-described PSGen 13 nucleic acid in a suitable carrier. For therapeutic uses, the present invention provides for a therapeutic amount of a PSGen 13 nucleic acid, for example as comprised in expressible form in a vector, in a suitable pharmaceutical carrier.

The present invention also provides for a procaryotic or eucaryotic host cell containing a PSGen 13 nucleic acid, as set forth above, for example as comprised in-a vector. The host cell may be a procaryotic or eucaryotic cell, including but not limited to a bacterial cell, a yeast cell, an insect cell, or a mammalian cell. The host cell may be a malignant or a non-malignant cell. In specific non-limiting embodiments of the invention, the host cell may be a human tumor cell, including, for example, a nasopharyngeal tumor cell, a thyroid tumor cell, a central nervous system tumor cell (e.g., a neuroblastoma, astrocytoma, or glioblastoma multiforme cell), a melanoma cell, an epithelial tumor cell, a non-epithelial tumor cell, a blood tumor cell, a leukemia cell, a lymphoma cell, a neuroblastoma cell, a cervical cancer cell, a breast cancer cell, a lung cancer cell, a prostate cancer cell, a colon cancer cell, a hepatic carcinoma cell, a urogenital cancer cell, an ovarian cancer cell, a testicular carcinoma cell, an osteosarcoma cell, a chondrosarcoma cell, a gastric cancer cell, or a pancreatic cancer cell.

5.2 PSGen 13 Proteins

The present invention provides for PSGen 13 proteins. In particular embodiments, the invention provides for a rPSGen 13 protein as depicted in FIG. 1 and having a sequence as set forth in SEQ ID NO:2, and substantially homologous proteins. In other particular embodiments, the invention provides for a HuPSGen 13 protein as depicted in FIG. 2 and having a sequence as set forth in SEQ ID NO:4, and substantially homologous proteins.

In additional embodiments, the present invention provides for a PSGen 13 protein encoded by a PSGen 13 nucleic acid as described above, and/or which cross-reacts with an antibody directed toward rPSGen 13 protein and/or HuPSGen 13 protein, as described below. Accordingly, in addition to human and rat PSGen 13 proteins, the present invention also provides for bovine, mouse protein, and non-human primate PSGen 13 proteins.

A PSGen 13 protein may be prepared from a natural source, may be chemically synthesized, or may be produced by recombinant DNA techniques. For example a PSGen 13 protein may be produced by expressing a PSGen 13 protein comprised in a suitable expression vector. A PSGen 13 protein may be expressed using standard techniques in a eucaryotic or a procaryotic expression system. Where a eucaryotic expression system is used, nucleic acid encoding a PSGen 13 protein, preferably comprised in a vector in expressible form, may be introduced into a eucaryotic cell by any standard technique, including transfection, transduction, electroporation, bioballistics, microinjection, etc.

The present invention provides for compositions comprising an above-described PSGen 13 protein in a suitable carrier. For therapeutic uses, the present invention provides for a therapeutic amount of a PSGen 13 protein in a suitable pharmaceutical carrier.

5.3 Antibodies to PSGen 13 Proteins

The invention provides for an antibody which binds specifically to a PSGen 13 protein described herein. In particular embodiments, the antibody binds specifically to a rPSGen 13 protein having a sequence as depicted in FIG. 1 and as set forth in SEQ ID NO:2. In another particular embodiment, the antibody binds to a HuPSGen 13 protein having a sequence as depicted in FIG. 2 and as set forth in SEQ ID NO:4. Such antibody may, in certain instances, cross-react with several PSGen 13 proteins from different species.

The antibody may be, for example but not by way of limitation, a human antibody, a murine antibody, a non-human primate antibody, a bovine antibody, a sheep antibody, a goat antibody, or a rat antibody. In specific non-limiting embodiments, the antibody may be a murine monoclonal antibody, a humanized monoclonal antibody, a human monoclonal antibody, a humanized primate monoclonal antibody, or a humanized rat monoclonal antibody.

According to the invention, a PSGen 13 protein, its fragments or other derivatives (e.g. histidine tagged protein), or analogs thereof, may be used as an immunogen to generate antibodies. Such antibodies include, but are not limited to, polyclonal, monoclonal, chimeric, single chain, Fab fragments, and a Fab expression library. In specific embodiments, antibodies which recognize rPSGen 13 or HuPSGen 13 are produced.

Various procedures known in the art may be used for the production of polyclonal antibodies which specifically bind to a PSGen 13 protein. For the production of antibody, various host animals can be immunized by injection with the native PSGen 13 protein, or a synthetic version, or derivative (e.g., fragment) thereof, including but not limited to rabbits, mice, rats, goats, etc. Various adjuvants may be used to increase the immunological response, depending on the host species, and including but not limited to Freund's (complete or incomplete) adjuvant, mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum.

For preparation of monoclonal antibodies directed toward a PSGen 13 protein, any technique which provides for the production of antibody molecules by continuous cell lines in culture may be used. Examples of such techniques include the hybridoma technique originally developed by Kohler and Milstein (1975, Nature 256:495-497), as well as the trioma technique, the human B-cell hybridoma technique (Kozbor et al., 1983, Immunology Today 4:72), and the EBV hybridoma technique to produce human monoclonal antibodies (Cole et al., 1985, in Monoclonal Antibodies and Cancer Therapy, Alan R. Liss, Inc., pp. 77-96). In an additional embodiment of the invention, monoclonal antibodies can be produced in germ-free animals utilizing recent technology (PCT/US90/02545). According to the invention, human antibodies may be used and can be obtained by using human hybridomas (Cote et al., 1983, Proc. Natl. Acad. Sci. U.S.A. 80:2026-2030) or by transforming human B cells with EBV virus in vitro (Cole et al., 1985, in Monoclonal Antibodies and Cancer Therapy, Alan R. Liss, pp. 77-96). Further, according to the invention, techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule specific for PSGen 13 together with genes from a human antibody molecule of appropriate biological activity may be used; such antibodies are within the scope of this invention.

According to the invention, techniques described for the production of single chain antibodies (U.S. Pat. No. 4,946,778) may be adapted to produce PSGen 13-specific single chain antibodies. An additional embodiment of the invention utilizes the techniques described for the construction of Fab expression libraries (Huse et al., 1989, Science 246:12751281) to allow rapid and easy identification of monoclonal Fab fragments with the desired specificity for PSGen 13 protein derivatives, or analogs.

Antibody fragments which contain the idiotype of the molecule can be generated by known techniques. For example, such fragments include but are not limited to: the F(ab′)₂, fragment which can be produced by pepsin digestion of the antibody molecule; the Fab′ fragments which can be generated by reducing the disulfide bridges of the F(ab′)₂, fragment, the Fab fragments which can be generated by treating the antibody molecule with papain and a reducing agent.

The invention provides for a composition which comprises an antibody described herein and a carrier. In particular embodiments of the invention, the composition is a pharmaceutical composition.

An antibody according to the invention may be used, for example, in the purification of a PSGen 13 protein or in a method of diagnosis to detect or measure the amount of PSGen 13 protein present, where the amount of PSGen 13 protein would be decreased in a cell as it progresses toward the transformed phenotype.

5.4 Uses of PSGen 13

The present invention relates to the use of PSGen 13 to suppress the transformed phenotype of a malignant cell. Indices of the transformed phenotype include but are not limited to cell proliferation (growth), morphology, lack of contact inhibition, the increased expression of transformation associated genes, the decreased expression of differentiation-specific genes, capability of anchorage-independent growth, lack of onset of senescence or apoptosis, tendency to form tumors and metastasize, etc. The effect of PSGen 13 may be mediated by introducing a PSGen 13 nucleic acid into a malignant cell, in expressible form, and/or by introducing a PSGen 13 protein or a PSGen 13 activator substance (see below).

Accordingly, the invention provides for a method for inhibiting growth of a cancer cell which comprises contacting the cancer cell with a nucleic acid encoding a PSGen 13 protein, a PSGen 13 protein, or a PSGen 13 activator substance. A PSGen 13 activator substance increases the level of PSGen 13 present in the cell, for example a substance which induces transcription of the PSGen 13 gene by promoter activation, or which increases the half-life of PSGen 13 protein or mRNA. The cancer cell, in particular embodiments, is a human cancer cell. In specific, non-limiting embodiments, the invention provides for a method for inhibiting growth of a human cancer cell comprising exposing said cell to an effective amount of a rPSGen 13 or HuPSGen 13 nucleic acid (in expressible form) or a rPSGen 13 or HuPSGen 13 protein. For example, but not by way of limitation, the human cancer cell may be a nasopharyngeal tumor cell, a thyroid tumor cell, a central nervous system tumor cell (e.g., a neuroblastoma, astrocytoma, or glioblastoma multiforme cell), a melanoma cell, an epithelial tumor cell, a non-epithelial tumor cell, a blood tumor cell, a leukemia cell, a lymphoma cell, a neuroblastoma cell, a cervical cancer cell, a breast cancer cell, a lung cancer cell, a prostate cancer cell, a colon cancer cell, a hepatic carcinoma cell, a urogenital cancer cell, an ovarian cancer cell, a testicular carcinoma cell, an osteosarcoma cell, a chondrosarcoma cell, a gastric cancer cell, or a pancreatic cancer cell.

In related embodiments, the invention provides for a method for treating a subject suffering from a cancer, comprising administering, to the subject, a therapeutically effective amount of a nucleic acid encoding a PSGen 13 protein, a PSGen 13 protein, or a PSGen 13 activator substance. The subject, in specific non-limiting embodiments, is a human. In particular non-limiting embodiments, the invention provides for a method for treating a human subject suffering from a cancer comprising administering to said subject a therapeutically effective amount of a rPSGen 13 or HuPSGen 13 nucleic acid (in expressible form) or rPSGen 13 or HuPSGen 13 protein. For example, the cancer to be treated may be a nasopharyngeal tumor, a thyroid tumor, a central nervous system tumor (e.g., a neuroblastoma, astrocytoma, or glioblastoma multiforme), melanoma, a vascular tumor, a blood vessel tumor (e.g., a hemangioma, a hemangiosarcoma), an epithelial tumor, a non-epithelial tumor, a blood tumor, a leukemia, a lymphoma, a neuroblastoma, a cervical cancer, a breast cancer, a lung cancer, a prostate cancer, a colon cancer, a hepatic carcinoma, a urogenital cancer, an ovarian cancer, a testicular carcinoma, an osteosarcoma, a chondrosarcoma, a gastric cancer, or a pancreatic cancer.

PSGen 13 nucleic acids may be introduced by any method known in the art which may utilize, for example, but not by way of limitation, vector mediated entry (e.g., infection), liposomes (e.g. DC-cholesterol liposomes, cationic liposomes, liposomes containing Sendai virus coat protein), imidazolium lipids (see, for example, U.S. Pat. No. 6,245,520 by Wang et al., issued Jun. 12, 2001), cationic lipids (see, for example, U.S. Pat. No. 6,235,310 by Wang et al., issued May 22, 2001), lipofection, asialoglycoprotein poly(L)lysine complexes, and microbubbles (see, for example, U.S. Pat. No. 6,245,747 by Porter et al., issued Jun. 12, 2001). A PSGen 13 protein may be introduced into a cell by any method known in the art, including methods which utilize formulations such as liposomes, microspheres, or vehicles that facilitate pinocytosis or phagocytosis.

The subject may be administered a therapeutically effective amount of a PSGen 13 gene or protein by any suitable route, including intra-tumor instillation, intravenous, intraarterial, intrathecal, intramuscular, intradermal, subcutaneous, etc. A therapeutically effective amount of these agents produces one or more of the following results: a decrease in tumor mass, a decrease in cancer cell number, a decrease in serum tumor marker, a decrease in tumor metastasis, a decrease in vascularization, a decrease in perfusion, a decreased rate of tumor growth, improved clinical symptoms, and/or increased patient survival. The cancer may be first treated surgically to de-bulk the tumor mass, if appropriate.

5.4.1 Identifying Potential Target Cells

It may be desirable to evaluate whether a particular cancer may be a suitable target for the methods of the present invention. Such an evaluation may be made, for example, by introducing a PS Gen 13 gene (in expressible form) or protein into a test cancer cell and determining whether the transformed phenotype of that cell is suppressed, for example, but not by way of limitation, by testing the proliferative capabilities of the cell and/or its ability to form colonies in soft agar.

In section 6, below, data is presented indicating that PSGen 13 suppresses the activity of the PEG3 promoter, a promoter associated with the progression associated gene PEG3 (see, for example, the PEG-3 promoter, as described in International Patent Application No. PCT/US99/07199, Publication No. WO 99/49898 (published in English on Oct. 7, 1999, incorporated by reference, and GenBank Accession No. AF351130). Activity of the promoter was monitored using the luciferase reporter gene. Based on this discovery, the present invention provides for a method for identifying putative PSGen 13 therapeutic targets by determining whether a cancer cell exhibits an increased level of PEG3. The methods of the invention may further comprise determining whether PSGen 13 can decrease PEG3 expression, for example by suppressing its promoter activity. Such activity may be monitored, in specific embodiments, using a PEG3 promoter/reporter gene construct, such as, but not limited to, the PEG3-Prom/luciferase gene reporter system exemplified herein.

In section 6, below, additional data is presented indicating that PSGen 13 suppresses the activity of the VEGF promoter, a promoter associated with the expression of Vascular Endothelial Growth Factor and new blood vessel formation (“angiogenesis”). Activity of the promoter was monitored using the luciferase reporter gene. Based on this discovery, the present invention provides for a method for identifying putative PSGen 13 therapeutic targets by determining whether a cancer cell exhibits an increased level of VEGF. The methods of the invention may further comprise determining whether PSGen 13 can decrease VEGF expression, for example by suppressing its promoter activity. Such activity may be monitored, in specific embodiments, using a VEGF promoter/reporter gene construct, such as, but not limited to, the VEGF-Prom/luciferase gene reporter system exemplified herein. Alternatively, tumors particularly associated with angiogenesis, such as vascular tumors such as melanoma or hemangiomas, may be suitable targets for PSGen 13-mediated treatments.

As set forth in section 8 below, HuPSGen 13 has been mapped to the chromosomal region 6q23.2-6q23.3. A cancer cell bearing a deletion in this region may be particularly sensitive to the transformation suppressive effects of HuPSGen 13. Accordingly, the present invention provides for a method for identifying a human cancer cell target for treatment with HuPSGen 13 comprising detecting a deletion in chromosomal region 6q23.2-6q23.3.

5.4.2 Inhibiting Angiogenesis

The invention provides for a method for inhibiting angiogenesis associated with tumor growth in a subject which comprises administering to the subject a pharmaceutical composition comprising a nucleic acid encoding a PSGen 13 protein, a PSGen 13 protein, or a PSGen 13 activator compound in a sufficient amount so as to inhibit angiogenesis associated with tumor growth in the subject. In particular non-limiting embodiments, the subject is a human, and the method of treatment comprises administering a therapeutically effective amount of rPSGen 13 or HuPSGen 13 nucleic acid, in expressible form, or of rPSGen 13 or HuPSgen 13 protein. In non-limiting embodiments, it may be desirable to administer a PSGen 13 nucleic acid or protein together with another molecule which has anti-angiogenic activity, such as angiostatin or thalidomide.

5.4.3 Diagnostic Methods

The present invention further provides for diagnostic methods which utilize the chromosomal location of HuPSGen 13, 6q23.2-6q23.3.

In one set of embodiments, the invention provides for a method of identifying an individual with an increased risk of developing a cancer, comprising detecting, in the individual, a deletion in chromosomal region 6q23.2-6q23.3. In specific non-limiting embodiments, the increased risk refers to the development of a cancer selected from the list including pancreatic cancer, papillary serous carcinoma of the peritoneum, hepatocellular carcinoma, large B cell lymphoma, prostate cancer, breast cancer, gastric cancer, and B cell non-Hodgkins lymphoma.

In another set of embodiments, the invention provides for a method of detecting progression of a cancer in a subject (i.e., the development of a more malignant phenotype) comprising detecting, in the individual, a deletion in chromosomal region 6q23.2-6q23.3. In specific non-limiting embodiments, the progression occurs in a cancer selected from the list including pancreatic cancer, papillary serous carcinoma of the peritoneum, hepatocellular carcinoma, large B cell lymphoma, prostate cancer, breast cancer, gastric cancer, and B cell non-Hodgkins lymphoma.

Deletions may be detected using any method known in the art, including but not limited to restriction fragment length polymorphism analysis.

### 6. EXAMPLE: PROGRESSION SUPPRESSED GENE 13 (PSGen 13) INHIBITS THE TRANSFORMED STATE IN RODENT AND HUMAN CANCER CELLS

Summary. A full-length rodent PSGen 13 gene was generated, placed in an expression vector and stably transfected into a progressed rodent transformed cell line, E11-NMT, and the DU-145 human prostate carcinoma cell line. A series of random single cell clones were isolated and evaluated for expression of the transformed state as documented by their ability to grow in an anchorage independent manner. Specific E11-NMT and DU-145 clones transfected with the rate PSGen 13 expression vector, but not the control expression vector lacking the rat PSGen 13 gene, were inhibited in their ability to grow in an anchorage independent manner. In the case of E11-NMT transformants transfected with rat PSGen 13, transcription of a specific progression elevated gene, PEG-3, and the vascular endothelial growth factor (VEGF) gene were inhibited. Using the rat PSGen 13 sequence, a human PSGen 13, HuPSGen 13, gene with significant DNA and protein sequence homology, 75 and 94%, respectively, to the rat PSGen 13 gene was identified. The studies described below demonstrate that PSGen 13 is a suppressor of the cancer phenotype and suppression may involve changes in the expression of cancer progression inducing and angiogenesis stimulating genes.

6.1 Materials and Methods

Cell cultures and agar growth assays. E11 is a single cell clone of H5ts125-transformed Sprague-Dawley secondary RE cells (Fisher et al., 1978, Proc. Natl. Acad. Sci. U.S.A. 75:2311-2314). E11-NMT is a subclone of E11 cells derived from a nude mouse tumor induced by the E11 cell line (Babiss et al., 1985, Science 228:1099-1101). R12 is a Ha-ras oncogene transformed E11 clone (Duigou et al., 1989, NY Acad Sci. 567:302-306). F1 and F2 are suppressed somatic cell hybrids with a flat morphology that were formed between E11-NMT and CREF cells (Duigou et al., 1990, Mol. Cell. Biol. 10:2027-2034). R1 and R2 are progressed somatic cell hybrids with a round morphology that were created by fusing E11-NMT and CREF cells (Id.). E11-HPV E6/E7 is a clone of E11 cells transformed with E6/E7 gene region of human papilloma virus type 18 (Su et al., 1997, Proc. Natl. Acad. Sci. U.S.A. 94:9125-9130). E11×E11-NMT A6 and 3b are independent somatic cell hybrid clones formed between E11 and E11-NMT cells that do not display the progression phenotype (Reddy et al., 1993, in Chromosome and Genetic Analysis, Methods in Molecular Genetics, Adolph, ed., Academic Press, Inc., Orlando, Fla. pp. 68-102). E11×E11-NMT cells that do not display the progression phenotype (Id.). E11×E11-NMT A6TD is a progressed somatic cell hybrid formed by isolating a tumor induced in a nude mouse by the E11×E11-NMT A6 somatic cell hybrid (Su et al., 1997, Proc. Natl. Acad. Sci. U.S.A. 94:9125-9130; Reddy et al., 1993, in Chromosome and Genetic Analysis, Methods in Molecular Genetics, Adolph, ed., Academic Press, Inc., Orlando, Fla. pp. 68-102). E11×E11-NMT IIa is a somatic cell hybrid formed between E11 and E11-NMT that exhibits the progression phenotype (Reddy et al., 1993, in Chromosome and Genetic Analysis, Methods in Molecular Genetics, Adolph, ed., Academic Press, Inc., Orlando, Fla. pp. 68-102). E11-NMT Aza B1 and Aza C1 are independent clones of E11-NMT cells treated with 5-azacytidine and displaying suppression in the progression phenotype (Su et al., 1997, Proc. Natl. Acad. Sci. U.S.A. 94:9125-9130; Reddy et al., 1993, in Chromosome and Genetic Analysis, Methods in Molecular Genetics, Adolph, ed., Academic Press, Inc., Orlando, Fla. pp. 68-102). CREF is a specific immortal non-transformed and non-tumorigenic clone of Fischer rat embryo fibroblast cells (Fisher et al., 1982, Proc. Natl. Acad. Sci. U.S.A. 79:3527-3531). DU-145 is a hormone refractive human prostate carcinoma cell line (Jiang et al., 1996, Proc. Natl. Acad. Sci. U.S.A. 93:9160-9165). All cultures were grown in Dulbecco's modified Eagle's medium (DMEM) supplemented with 5% FBS (DMEM-5) at 37° C. in a humidified 5% CO₂/95% air incubator. Anchorage independent growth assays were performed as described previously by seeding variable numbers of cells in 0.4% noble agar containing medium on a base layer of 0.8% noble agar containing medium (Kang et al., 1998, Proc. Natl. Acad. Sci. U.S.A. 95:13788-13793; Fisher et al., 1979, Cancer Res. 39:3051-3057; Fisher et al., 1979, Nature 281:591-594; Fisher et al., 1979, Cell 18:695-705; Babiss et al., 1985, Science 228:1099-1101; Su et al., 1997, Proc. Natl. Acad. Sci. U.S.A. 94:9125-9130; Su et al., 1999, Proc. Natl. Acad. Sci. U.S.A. 96:15115-15120; Fisher et al., 1978, Proc. Natl. Acad. Sci. U.S.A. 75:2311-2314).

Construction of PSGen 13 expressing E11-NMT clones. E11-NMT and DU-145 cells were transfected, as previously described (Su et al., 1997, Proc. Natl. Acad. Sci. U.S.A. 94:9125-9130), with a pcDNA3.1 (+) expression vector (containing a neomycin resistance gene) lacking or having the complete rPSGen 13 gene. Briefly, 1×10⁵ cells were seeded in 10-cm tissue culture plates, and 6 hr later 10 g of purified pcDNA3.1 (+) vector or a rat PSGen 13/pcDNA3.1 (+) construct was incubated with 30 μl of Lipofectamine (Gibco BRL) and this mixture was added to cells for 8 hr. The next day the media was changed with the addition of 500 μg/ml of G418 and media was changed 2× per week for three weeks. G418-resistant colonies were isolated using cloning cylinders and maintained as independent cell lines, referred to as NMT-PSG13 clones (cl 3, 5, 6, 7, 8, 9, 10, 11 and 12) and DU-PSG13 clones (cl 11, 12, 13, 14, 15 and 17), in complete media containing 100 μg/ml of G418. Additionally, NMT-vector and DU-145-Vec clones were isolated and maintained as independent cell lines in complete media containing 10 μg/ml of G418.

Northern blotting assays. Total cellular RNA was isolated by the guanidinium/phenol extraction method and Northern blotting was performed as described (Su et al., 1997, Proc. Natl. Acad. Sci. U.S.A. 94:9125-9130). Fifteen micrograms of RNA were denatured and electrophoresed in 1.2% agarose gels with 3% formaldehyde, transferred to nylon membranes and hybridized sequentially with a ³²P-labeled rPSGen 13 cDNA probe, the blot was stripped and then reprobed with a ³²P-labeled GAPDH cDNA probe as described previously (Id.). Following hybridization, the filters were washed and exposed for autoradiography.

Promoter Analyses. To evaluate the activity of the full length rat PEG-3 promoter (PEG-Prom) and VEGF-Promoter constructs, cells (E11, E11-NMT and NMT-PSGen13) clones, were seeded at 2×10⁵/35-mm tissue culture plate and about 24 h later transfected with 5 μg of the PEG-Prom-luciferase or VEGF-Prom-Luciferase constructs plus 1 μg of SV40-β-gal Vector (Promega) mixed with 10 μl of Lipofectamine Reagent (Gibco) in 200 μl of serum-free media. After 20 min at RT, 800 μl of serum-free media were added resulting in a final volume of 1 ml. The transfection mixture was removed after 14 hr and the cells were washed 3× with serum-free media and incubated at 37° C. for an additional 48 hr in complete growth media. Cells were harvested and lysed to make extracts (Gopalkrishan et al., 1999, Nucl. Acids Res. 27:4775-4782) utilized in β-gal and Luciferase reporter assays. Luminometric determinations of Luciferase and β-gal activity were performed using commercial kits (Promega and Tropix, respectively). For Luciferase assays, 10 μl of cell lysate were mixed with 40 μl of Luciferase Assay substrate (Promega). For β-gals assays, 10 μl of the cell lysate were mixed with 100 μl of diluted Galecton-Plus with 150 μl of Accelerator (Tropix). Promoter analysis data were collected a minimum of three times using triplicate samples for each experimental point and the data was standardized with the β-gal data.

Cloning a full length rat PSGen 13 and a HuPSGen 13 cDNA. An original rat PSGen 13 EST was identified using RSDD and reverse Northern hybridization as a gene displaying elevated expression in E11 versus E11-NMT cells (Kang et al., 1998, Proc. Natl. Acad. Sci. U.S.A. 95:13788-13793). A full length open reading frame (ORF) of rat PSGen 13 was cloned using the complete open reading frame (C-ORF) approach with gene specific primers and electronic data mining based on the EST sequence. Primers used for C-ORF were PSGen13-R2 (TCG CTT CTC ACT TTG ACG GAG TGT CAA G) (SEQ ID NO: 7) and PSGen13-R2 Nested (TGT CAA GTG TGG CAG AGA CTA AGA ATG G) (SEQ ID NO: 8). In addition, full length rPSGen 13 and HuPSGen 13 cDNA clones were identified by sequence comparison of the rat PSGen 13 EST sequence with GenBank by BLAST. Selected clones (ATCC #2005777 from rat PSGen 13 and ATCC #2525262 for HuPSGen 13) were procured (Research Genetics) and sequenced.

### 6.2. EXPERIMENTAL RESULTS

Sequence Informatics of PSGen 13. The cloned full length rat PSGen 13 cDNA consists of 780 bp excluding the poly(A) tail. A poly(A) signal (AATAAA) is located at position 763 (FIG. 1; SEQ ID NO: 1). There is an in-frame stop codon at 86 bp (not present in HuPSGen 13), and the ORF starts at the first ATG at 170 bp and spans to 415 bp. The rPSGen 13 cDNA encodes a protein with predicted 81 amino acids (FIG. 1; SEQ ID NO:2) of calculated molecular weight of 9 kDa with a pI of 5.52. Protein sequence analysis did not indicate hydrophobic patches for membrane spanning regions or signal peptide sequences characteristic of secretory proteins. Motif and pattern analysis also failed to identify sequence homologies with previously reported genes, information that is useful in providing potential insights into the biological function and or mode of action of rat PSGen 13. Based on this observation, rPSGen 13 appears to encode a novel class of proteins.

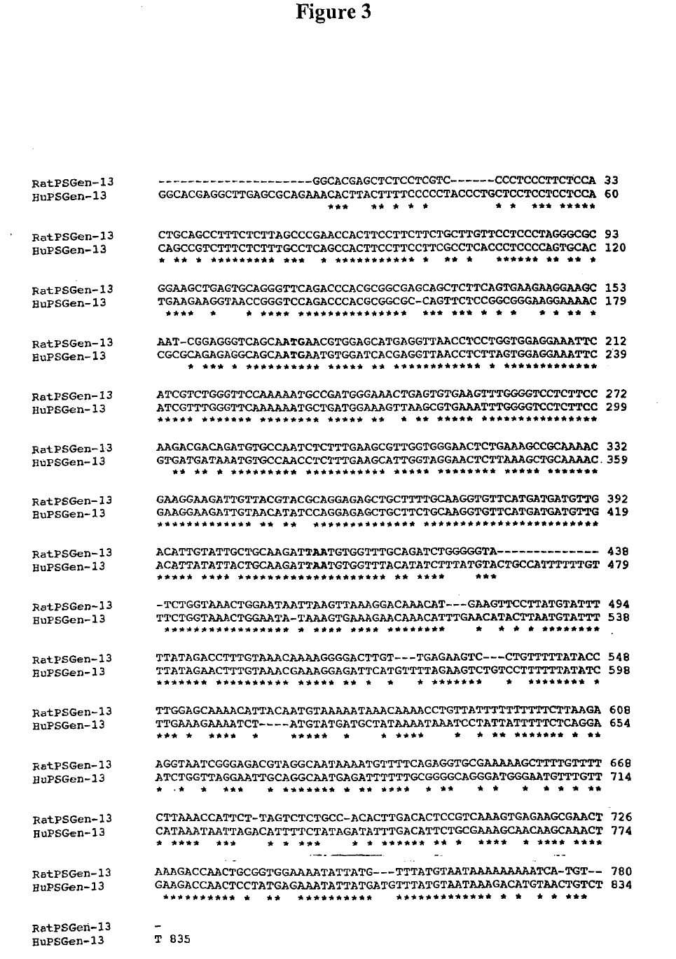

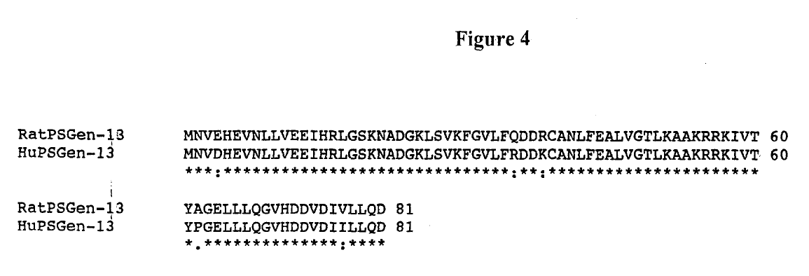

A human homologue of rPSGen 13, HuPSGen 13, was electronically cloned by analyzing sequences reported in the GenBank data base (FIG. 2; SEQ ID NO: 3). HuPSGen 13 is 75% identical to rPSGen 13 at the nucleotide level, but 94% identical to rPSGen 13 at the protein level (79/81 residues) (FIGS. 3 and 4). Of the five residues that are distinct in HuPSGen 13, three of them (D at 4, K at 38 and I at 77) are conserved substitutions of rPSGen 13 (E at 4, R at 38 and V at 77, respectively), which suggests strong conservation in functionality. Furthermore, sequence identity of the HuPSGen 13 with rPSGen 13 protein coding sequence is 87% at the nucleotide level. Both 5′ and 3′ untranslated regions display 68.7% and 68.3% identity, respectively, and are more diverse between rPSGen 13 and HuPSGen 13 than the ORF, which is not uncommon between interspecies homologues. Considering the degree of conservation in the ORF and resulting protein sequence, HuPSGene is an orthologue of rPSGen 13. The cloned HuPSGen 13 cDNA consists of 835 bp excluding the poly(A) tail and a canonical poly(A) signal was observed at 814 bp. Although an in-frame stop codon was not present, the ORE of HuPSGen 13 starts at the first ATG (197 bp) and runs through 442 bp. HuPSGen 13 encodes 81 amino acids of calculated Mol. Wt. of 9 kDa with a pI of 5.86. As in r PSGen 13, computational protein sequence analysis did not yield any known functional motifs.

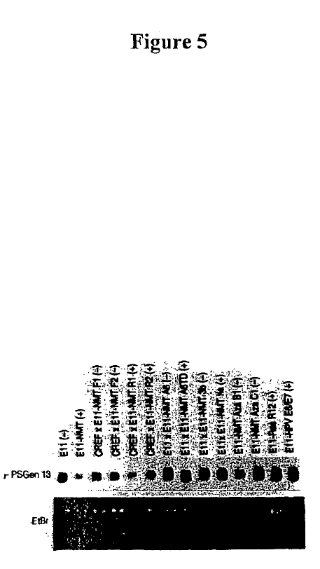

rPSGen 13 Suppresses Anchorage Independent Growth in E11-NMT and DU-145 Cells. rPSGen 13 was identified using RSDD as a gene displaying elevated expression in E11 cells versus E11-NMT cells (Kang et al., 1998, Proc. Natl. Acad. Sci. U.S.A. 95:13788-13793). Using a panel of rodent cell lines displaying either a progressed phenotype (+), as indicated by elevated growth in agar and short tumor latency times, or unprogressed (−), as indicated by reduced growth in agar and extended tumor latency times, a direct correlation between reduced rPSGen 13 expression and the progression phenotype was found (FIG. 5, from Kang et al., 1998, Proc. Natl. Acad. Sci. U.S.A. 95:13788-13793). The level of rPSGen 13 was elevated in E11, CREF×E11-NMT F1 and F2, E11×E11-NMT A6, and E11-NMT AZA B 1 cells which do not display the progression phenotype. In contrast, rPSGen 13 expression was lower in progressed E11-NMT, CREF×E11-NMT R1 and R2, E11×E11-NMT A6TD, E11×E11-NMT IIa, E11-Ras R12 and E11-HPV E6/E7 cells. These findings documented a potential inverse relationship between expression of the progression phenotype and rat PSGen 13 expression. They also raised the possibility that PSGen 13 might play a functional role in the progression process.

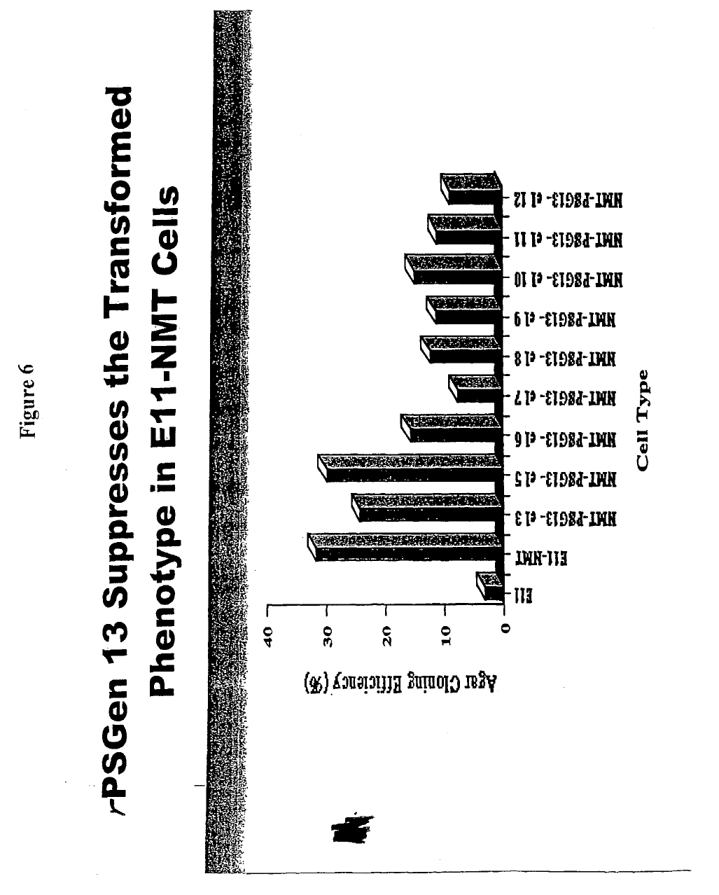

r PSGen 13 was cloned into an expression vector and stably transfected into E11-NMT and DU-145 cells. Random clones were isolated and evaluated for their ability to form macroscopic colonies when seeded in semi-solid agar (Fisher et al., 1979, Cancer Res. 39:3051-3057; Fisher et al., 1979, Nature 281:591-594; Fisher et al., 1979, Cell 18:695-705). E11 cells grow with a low: efficiency in agar, whereas progressed E11-NMT cells display elevated growth in agar, forming more and larger colonies than E11 cells (FIG. 6) (Id.). Analysis of the random NMT-PSGen 13 clones, designated as NMT-PSG13 cl number, indicated specific clones displaying no significant reduction in anchorage independence, i.e., NMT-PSG13 cl 3 and 5, and clones displaying reduced agar cloning efficiencies similar to E11 cells, i.e., NMT-PSG13 cl 7 and 12. In addition, a number of clones were also identified that displayed a significant reduction in anchorage independence versus E11-NMT cells, but less growth suppression than NMT-PSG13 cl 7 and 12, including NMT-PSG13 cl 6, 8, 9, 10, and 11.

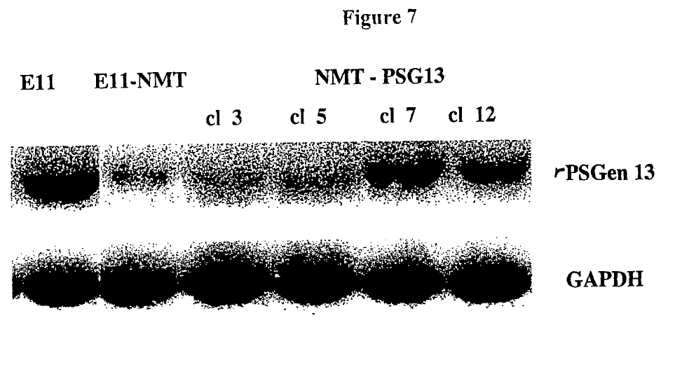

To confirm that suppression of anchorage independence was associated with increased expression of rPSGen 13, total RNA was isolated from E11, E11-NMT and NMT-PSG13 cl 3, 5, 7 and 12 and evaluated by Northern blotting (FIG. 7). This experiment documented expression of PSGen 13 in E11 and NMT-PSG13 cl 7 and 12, but not in E11-NMT or NMT-PSG13 cl 3 and 5. These results demonstrate that forced expression of rPSGen-13 in E11-NMT cells resulted in a suppression of anchorage independent growth, a marker of the progression phenotype in this transformational model system.

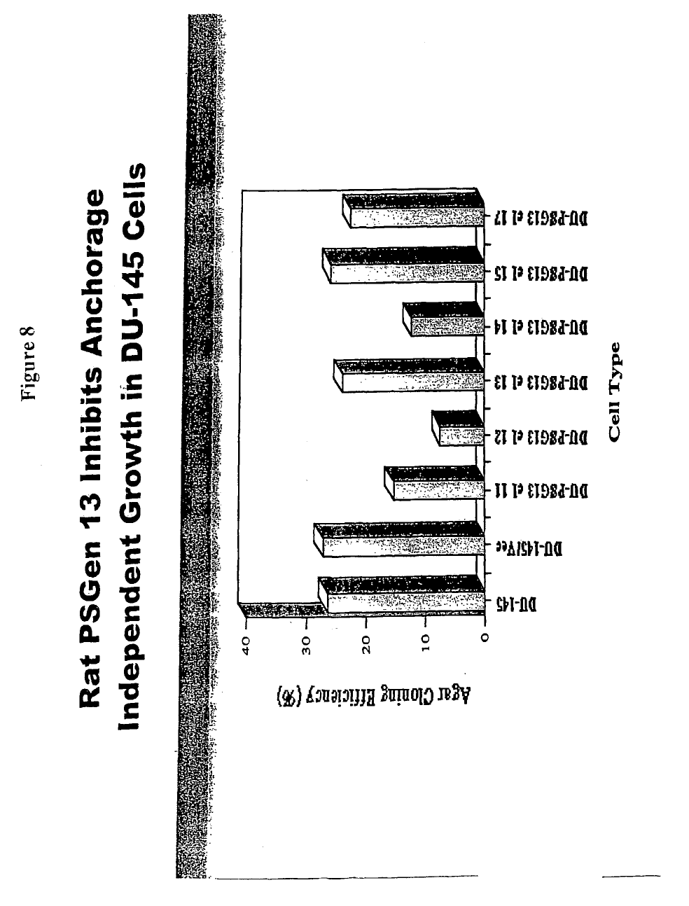

To determine if the rPSGen 13 gene could effect the transformed phenotype in human cancer cells, a full-length rPSGen 13 cDNA was transfected into DU-145 human prostate cancer cells and random clones were isolated. The clones were then evaluated for anchorage independent growth (FIG. 8). Several clones were identified, including DU-PEG13 cl 11, 12 and 14, that displayed a significant reduction in anchorage independent growth in comparison with DU-145 cells. Additional clones, including a vector transfected clone and DU-PEG13 cl 13, 15 and 17 displayed similar cloning efficiencies in agar as untransfected DU-145 cells. These results demonstrate that rat PSGen 13 can also suppress the transformed phenotype in human cancer cells, thereby indicating a more general inhibitory capacity of this gene product in tumor cells.

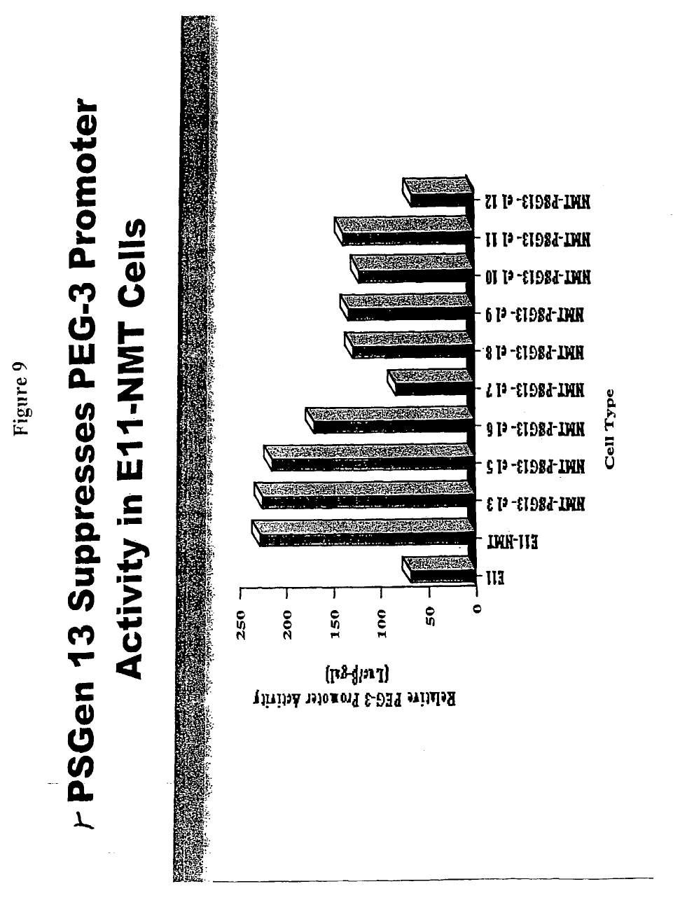

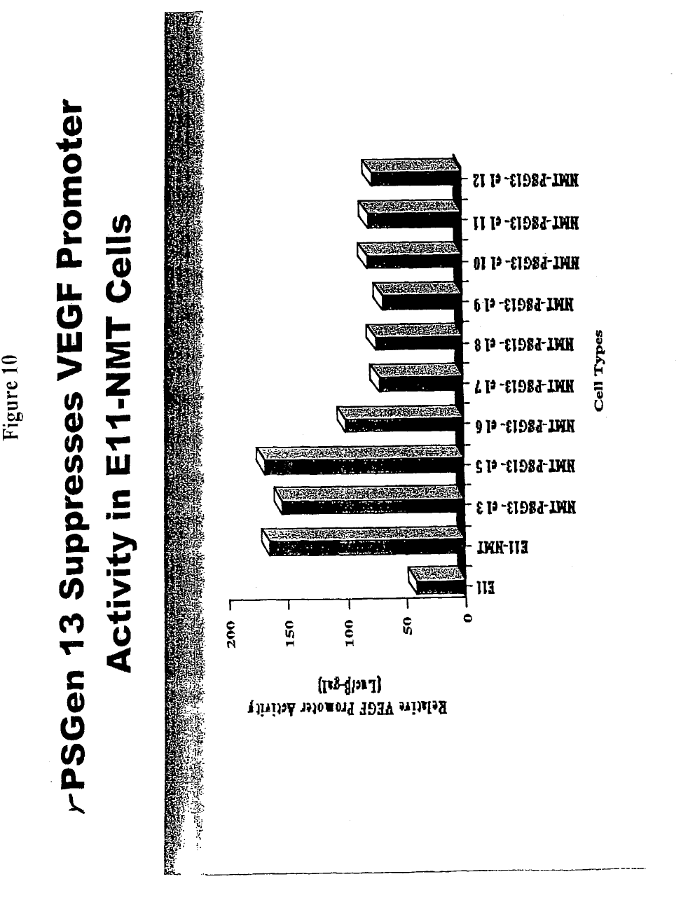

Rat PSGen 13 Inhibits Transcriptional Activity in E11-NMT Cells. Previous studies indicated that E11-NMT cells display elevated transcription of the PEG-3 and VEGF genes as compared to E11 cells (Su et al., 1999, Proc. Natl. Acad. Sci. U.S.A. 96:15115-15120). These changes in gene expression are believed to be important determinants of the aggressive progressed cancer phenotype of E11-NMT versus E11 cells (Id.). To determine if rPSGen 13 expression can effect these important progression related genes, the transcriptional activities of the PEG3 promoter linked to the luciferase reporter gene (“PEG-Prom-Luciferase”) and the VEGF promoter linked to the luciferase reporter gene (“VEGF-Prom-Luciferase”) constructs were evaluated in E11, E11-NMT and the different NMT-PSG13 clones (FIGS. 9 and 10). As previously reported (Id.), the PEG-Prom and VEGF-Prom were more active in E11-NMT than in E11 cells. Both NMT-PSG13 cl 7 and 12 displayed a reduction in PEG-Prom activity similar to that of E11 cells (FIG. 9). In contrast, when compared with E11-NMT cells the levels of PEG-Prom-Luciferase activity were unchanged in NMT-PSG13 cl 3 and 5, which do not express elevated rPSGen 13 mRNA (FIG. 7). Although of lesser magnitude than observed in NMT-PSG13 cl 7 and 12, reductions in the level of PEG-Prom activity were also apparent in NMT-PSG13 cl 6 (smallest reduction), 8, 9, 10 and 11. In the case of the VEGF-Prom, no change in promoter activity was apparent in NMT-PSG13 cl 3 and 5 versus E11-NMT cells (FIG. 10). However, a similar reduction in VEGF-Prom activity, approaching that observed in E11 cells, was apparent in NMT-PSG13 cl 7, 8, 9, 10, 11 and 12. These results suggest that expression of PSGen 13 can regulate expression of both PEG-3 and VEGF. It is not presently known if this regulation of the transcriptional activity of either of these two genes occurs by a direct or indirect mechanism.

### 6.3 CONCLUSIONS

The RSDD approach has successfully identified a novel gene, rPSGen 13, that can functionally regulate cancer progression in both rodent and human tumor cells. Evidence is presented that forced expression of rPSGen 13 in rodent tumor cells displaying an aggressive cancer phenotype, E11-NMT, resulted in a suppression of the cancer phenotype as monitored by a reduction in anchorage independent growth. Similarly, when overexpressed in a human prostate cancer cell line (DU-145), rPSGen 13 also induced an inhibition in anchorage independence. The mechanism by which PSGen 13 induces its cancer growth suppression properties is not known. However, in the E 11-NMT model forced expression of rPSGen 13 directly correlated with a suppression in the transcriptional activities of two significant cancer progression regulating genes, PEG-3 and VEGF.

Based on sequence homology, a human PSGen 13 gene, HuPSGen 13, has been isolated. This gene is highly homologous to the rat PSGen 13 gene, 75% and 94% on a nucleotide and protein level, respectively. As shown in the following section, the HuPSGen 13 also has been demonstrated to suppress the transformed phenotype.

### 7. EXAMPLE: HUMAN PSGEN 13 SUPPRESSES THE TRANSFORMED PHENOTYPE

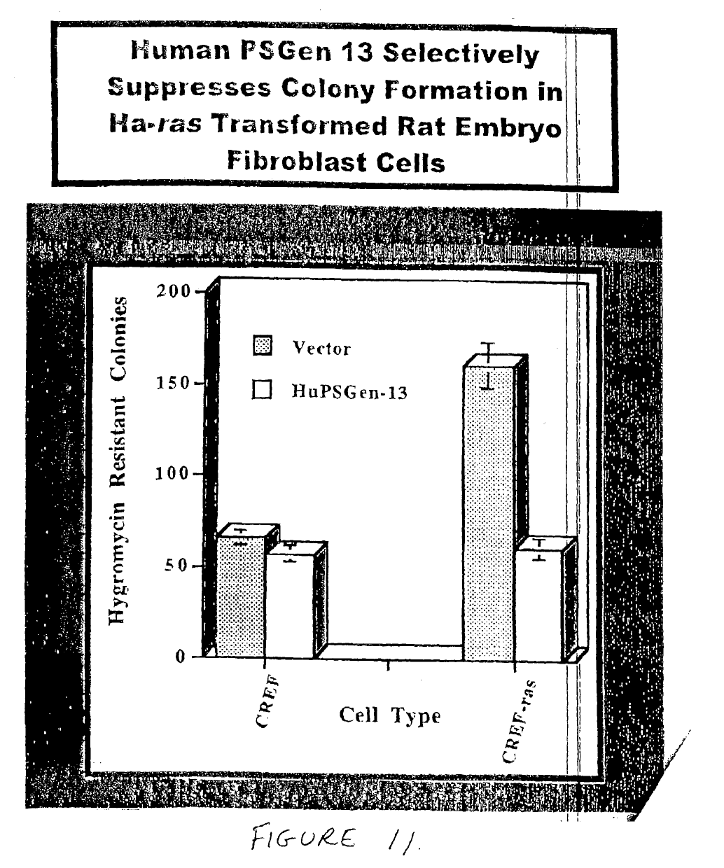

HuPSGen 13 suppressed the transformed phenotype of transformed rat embryo cells. Both CREF cells and CREF cells transformed by Ha-ras (“CREF-ras” cells) were transfected with either empty vector or vector containing HuPSGen 13. The vector also contained a hygromycin resistance gene. Transfectants were selected for hygromycin resistance and colony formation in monolayer culture was assessed. The results are shown in FIG. 11, which depicts the results of triplicate plates, ±S.D. This data demonstrates that HuPSGen 13 exerted a selective inhibitory effect on colony formation in the CREF-ras transformed rat embryo fibroblast cells. Moreover, colony-forming efficiency in transfected Ha-ras transformed cells was found to revert back to the level observed in untransformed transfected CREF cells.

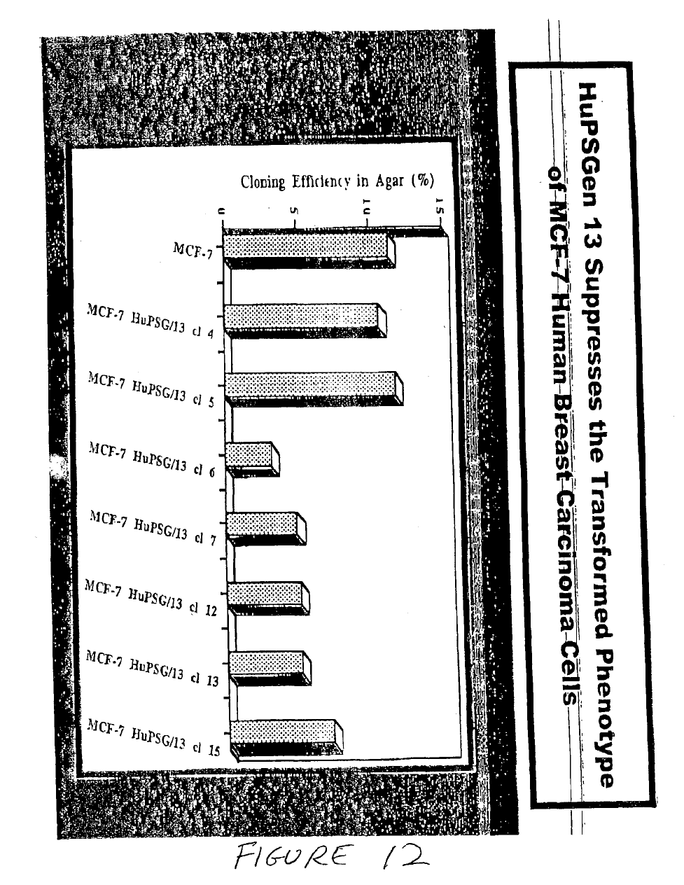

HuPSGen 13 suppressed the transformed phenotype of human breast cancer cells. Cells of the human breast carcinoma cell line MCF-7 were transfected with HuPSGen 13 contained in a vector that further comprised a hygromycin resistance gene. Transfectants were then selected in hygromycin and isolated clones were evaluated for anchorage independent growth in agar. As shown in FIG. 12, although some HuPSGen 13 transfected cell lines exhibited a cloning efficiency similar to untransfected MCF-7 cells (clones 4 and 5), the cloning efficiency was decreased substantially in human PSGen 13 transfected MCF-7 clones 6, 7, 12, 13 and 15.

### 8. EXAMPLE: CHROMOSOMAL LOCATION OF HUPSGEN 13

The chromosomal location of HuPSGen 13 has been mapped to chromosomal locus 6q23.2-6q23.3. Deletions in this region have been noted in a variety of cancers, including pancreatic cancer, papillary serous carcinoma of the peritoneum, hepatocellular carcinoma, large B cell lymphoma, prostate cancer, breast cancer, gastric cancer, and B cell non-Hodgkins lymphoma.

Throughout this application, various publications are referenced by author and date within the text. All patents, patent applications, GenBank sequences and publications cited herein, including the parent application, U.S. patent application Ser. No. 09/648,310, are hereby incorporated by reference into this application in order to more fully describe the state of the art as known to those skilled therein as of the date of the invention described and claimed herein.

What is claimed is:

1. An isolated nucleic acid encoding a Progression Suppressed Gene 13 protein, operably linked to a promoter element active in eucaryotic cells.

2. An isolated nucleic acid encoding a rat Progression Suppressed Gene 13 protein and having a nucleic acid sequence as set forth in SEQ ID NO: 1, operably linked to a promoter element active in eucaryotic cells.

3. An isolated nucleic acid that hybridizes under stringent conditions to the nucleic acid of claim 2, operably linked to a promoter element active in eucaryotic cells.

4. An isolated nucleic acid encoding a human Progression Suppressed Gene 13 protein and having a nucleic acid sequence as set forth in SEQ ID NO:3, operably linked to a promoter element active in eucaryotic cells.

5. An isolated nucleic acid that hybridizes under stringent conditions to the nucleic acid of claim 4, operatively linked to a promoter element active in eucaryotic cells.

6. An isolated nucleic acid encoding a protein having an amino acid sequence as set forth in SEQ ID NO:2.

7. An isolated nucleic acid encoding a protein having an amino acid sequence as set forth in SEQ ID NO:4.

8. A vector comprising the nucleic acid of claim 1.

9. A vector comprising the nucleic acid of claim 2.

10. A vector comprising the nucleic acid of claim 3.

11. A vector comprising the nucleic acid of claim 4.

12. A vector comprising the nucleic acid of claim 5.

13. A vector comprising the nucleic acid of claim 6.

14. A vector comprising the nucleic acid of claim 7.

15. A host cell comprising the vector of claim 8.

16. A host cell comprising the vector of claim 9.

17. A host cell comprising the vector of claim 10.

18. A host cell comprising the vector of claim 11.

19. A host cell comprising the vector of claim 12.

20. A host cell comprising the vector of claim 13.

21. A host cell comprising the vector of claim 14.

22. The host cell of any one of claims 15-21, wherein the host cell is a tumor cell.

23. The host cell of claim 22, wherein the tumor cell is selected from the group consisting of a nasopharyngeal tumor cell, a thyroid tumor cell, a central nervous system tumor cell, a neuroblastoma cell, an astrocytoma cell, a glioblastoma multiforme cell, a melanoma cell, an epithelial tumor cell, a non-epithelial tumor cell, a blood tumor cell, a leukemia cell, a lymphoma cell, a neuroblastoma cell, a cervical cancer cell, a breast cancer cell, a lung cancer cell, a prostate cancer cell, a colon cancer cell, a hepatic carcinoma cell, a urogenital cancer cell, an ovarian cancer cell, a testicular carcinoma cell, an osteosarcoma cell, a chondrosarcoma cell, a gastric cancer cell, or a pancreatic cancer cell.

24. A pharmaceutical composition comprising a therapeutically effective amount of a nucleic acid according to any one of claims 1-7 in a suitable pharmaceutical carrier.

25. A pharmaceutical composition comprising a therapeutically effective amount of a vector according to any one of claims 8-14 in a suitable pharmaceutical carrier.

26. An isolated Progression Suppressed Gene 13 protein.

27. A composition comprising the Progression Suppressed Gene 13 protein of claim 26 together with a pharmaceutical carrier.

28. An isolated Progression Suppressed Gene 13 protein having an amino acid sequence as set forth in SEQ ID NO:2.

29. A composition comprising the Progression Suppressed Gene 13 protein of claim 28 together with a pharmaceutical carrier.

30. An isolated Progression Suppressed Gene 13 protein having an amino acid sequence as set forth in SEQ ID NO:4.

31. A composition comprising the Progression Suppressed Gene 13 protein of claim 30 together with a pharmaceutical carrier.

32. An antibody which specifically binds to the protein of claim 28.

33. An antibody which specifically binds to the protein of claim 30.

34. An isolated protein which binds to the antibody of claim 32.

35. An isolated protein which binds to the antibody of claim 33.

36. A method for suppressing the transformed phenotype of a cell, comprising administering, to the cell, an effective amount of a nucleic acid encoding a Progression Suppression Gene 13 protein, in expressible form.

37. The method of claim 36, wherein the nucleic acid has a sequence as set forth in SEQ ID NO: 1.

38. The method of claim 36, wherein the nucleic acid has a sequence as set forth in SEQ ID NO:3.

39. A method for inhibiting cancer cell growth, comprising administering to said cancer cell an effective amount of a nucleic acid encoding a Progression Suppression Gene 13 protein, in expressible form.

40. The method of claim 39, wherein the nucleic acid has a sequence as set forth in SEQ ID NO: 1.

41. The method of claim 39, wherein the nucleic acid has a sequence as set forth in SEQ ID NO:3.

42. A method of inhibiting angiogenesis in a tumor, comprising administering to the tumor an effective amount of a nucleic acid encoding a Progression Suppression Gene 13 protein, in expressible form.

43. The method of claim 42, wherein the nucleic acid has a sequence as set forth in SEQ ID NO: 1.

44. The method of claim 42, wherein the nucleic acid has a sequence as set forth in SEQ ID NO:3.

45. A method of treating cancer in a subject, comprising administering, to the subject, an effective amount of a nucleic acid encoding a Progression Suppression Gene 13 protein, in expressible form.

46. The method of claim 45, wherein the nucleic acid has a sequence as set forth in SEQ ID NO: 1.

47. The method of claim 45, wherein the nucleic acid has a sequence as set forth in SEQ ID NO:3.

48. A method for suppressing the transformed phenotype of a cell, comprising administering, to the cell, an effective amount of a Progression Suppression Gene 13 protein.

49. The method of claim 48, wherein the protein has a sequence as set forth in SEQ ID NO:2.

50. The method of claim 48, wherein the protein has a sequence as set forth in SEQ ID NO:4.

51. A method for inhibiting cancer cell growth, comprising administering to said cancer cell an effective amount of a Progression Suppression Gene 13 protein.

52. The method of claim 51, wherein the protein has a sequence as set forth in SEQ ID NO:2.

53. The method of claim 51, wherein the protein has a sequence as set forth in SEQ ID NO:4.

54. A method of inhibiting angiogenesis in a tumor, comprising administering to the tumor an effective amount of a Progression Suppression Gene 13 protein.

55. The method of claim 54, wherein the protein has a sequence as set forth in SEQ ID NO:2.

56. The method of claim 54, wherein the protein has a sequence as set forth in SEQ ID NO:4.

57. A method of treating cancer in a subject, comprising administering, to the subject, an effective amount of a Progression Suppression Gene 13 protein.

58. The method of claim 57, wherein the protein has a sequence as set forth in SEQ ID NO:2.

59. The method of claim 57, wherein the protein has a sequence as set forth in SEQ ID NO:4.

60. A method for suppressing the transformed phenotype of a breast cancer cell, comprising administering, to the cell, an effective amount of a nucleic acid encoding a Progression Suppression Gene 13 protein, in expressible form.

61. The method of claim 60, wherein the nucleic acid has a sequence as set forth in SEQ ID NO: 1.

62. The method of claim 60, wherein the nucleic acid has a sequence as set forth in SEQ ID NO:3.

63. A method for inhibiting breast cancer cell growth, comprising administering to said cancer cell an effective amount of a nucleic acid encoding a Progression Suppression Gene 13 protein, in expressible form.

64. The method of claim 63, wherein the nucleic acid has a sequence as set forth in SEQ ID NO: 1.

65. The method of claim 63, wherein the nucleic acid has a sequence as set forth in SEQ ID NO:3.

66. A method of inhibiting angiogenesis in a breast tumor, comprising administering to the tumor an effective amount of a nucleic acid encoding a Progression Suppression Gene 13 protein, in expressible form.

67. The method of claim 66, wherein the nucleic acid has a sequence as set forth in SEQ ID NO: 1.

68. The method of claim 66, wherein the nucleic acid has a sequence as set forth in SEQ ID NO:3.

69. A method of treating breast cancer in a subject, comprising administering, to the subject, an effective amount of a nucleic acid encoding a Progression Suppression Gene 13 protein, in expressible form.

70. The method of claim 69, wherein the nucleic acid has a sequence as set forth in SEQ ID NO:1.

71. The method of claim 69, wherein the nucleic acid has a sequence as set forth in SEQ ID NO:3.

72. A method for suppressing the transformed phenotype of a prostate cancer cell, comprising administering, to the cell, an effective amount of a nucleic acid encoding a Progression Suppression Gene 13 protein, in expressible form.

73. The method of claim 72, wherein the nucleic acid has a sequence as set forth in SEQ ID NO: 1.

74. The method of claim 72, wherein the nucleic acid has a sequence as set forth in SEQ ID NO:3.

75. A method for inhibiting prostate cancer cell growth, comprising administering to said cancer cell an effective amount of a nucleic acid encoding a Progression Suppression Gene 13 protein, in expressible form.

76. The method of claim 75, wherein the nucleic acid has a sequence as set forth in SEQ ID NO:1.

77. The method of claim 75, wherein the nucleic acid has a sequence as set forth in SEQ ID NO:3.

78. A method of inhibiting angiogenesis in a prostate tumor, comprising administering to the tumor an effective amount of a nucleic acid encoding a Progression Suppression Gene 13 protein, in expressible form.

79. The method of claim 78, wherein the nucleic acid has a sequence as set forth in SEQ ID NO:1.

80. The method of claim 78, wherein the nucleic acid has a sequence as set forth in SEQ ID NO:3.

81. A method of treating prostate cancer in a subject, comprising administering, to the subject, an effective amount of a nucleic acid encoding a Progression Suppression Gene 13 protein, in expressible form.

82. The method of claim 81, wherein the nucleic acid has a sequence as set forth in SEQ ID NO: 1.

83. The method of claim 81, wherein the nucleic acid has a sequence as set forth in SEQ ID NO:3.

84. A method for identifying a human cancer cell target for treatment with HuPSGen 13 comprising detecting a deletion in chromosomal region 6q23.2-6q23.3.

85. A method of identifying an individual with an increased risk of developing a cancer, comprising detecting, in the individual, a deletion in chromosomal region 6q23.2-6q23.3.

86. The method of claim 85, wherein the cancer is selected from the group consisting of pancreatic cancer, papillary serous carcinoma of the peritoneum, hepatocellular carcinoma, large B cell lymphoma, prostate cancer, breast cancer, gastric cancer, and B cell non-Hodgkins lymphoma.

87. A method of detecting progression of a cancer in a subject comprising detecting, in the individual, a deletion in chromosomal region 6q23.2-6q23.3.

88. The method of claim 87, wherein the cancer is selected from the group consisting of pancreatic cancer, papillary serous carcinoma of the peritoneum, hepatocellular carcinoma, large B cell lymphoma, prostate cancer, breast cancer, gastric cancer, and B cell non-Hodgkins lymphoma.


SEQUENCE LISTING (8 sequences)

SEQ ID NO: 1 - DNA, 780 bp, rattus norvegicus
ggcacgagctctcctcgtcccctcccttctccactgcagcctttctcttagcccgaaccacttccttcttctgcttgttcctccctagggcgcggaagctgagtgcagggttcagacccacgcggcgagcagctcttcagtgaagaaggaagcaatcggagggtcagcaatgaacgtggagcatgaggttaacctcctggtggaggaaattcatcgtctgggttccaaaaatgccgatgggaaactgagtgtgaagtttggggtcctcttccaagacgacagatgtgccaatctctttgaagcgttggtgggaactctgaaagccgcaaaacgaaggaagattgttacgtacgcaggagagctgcttttgcaaggtgttcatgatgatgttgacattgtattgctgcaagattaatgtggtttgcagatctgggggtatctggtaaactggaataattaagttaaaggacaaacatgaagttccttatgtatttttatagacctttgtaaacaaaaggggacttgttgagaagtcctgtttttataccttggagcaaaacattacaatgtaaaaataaacaaaacctgttattttttttttcttaagaaggtaatcgggagacgtaggcaataaaatgttttcagaggtgcgaaaaagcttttgttttcttaaaccattcttagtctctgccacacttgacactccgtcaaagtgagaagcgaactaaagaccaactgcggtggaaaatattatgtttatgtaataaaaaaaaatcatgt

SEQ ID NO: 2 - protein, 81 aa, rattus norvegicus
MNVEHEVNLLVEEIHRLGSKNADGKLSVKFGVLFQDDRCANLFEALVGTLKAAKRRKIVTYAGELLLQGVHDDVDIVLLQD

SEQ ID NO: 3 - DNA, 835 bp, homo sapiens
ggcacgaggcttgagcgcagaaacacttacttttccccctaccctgctcctcctcctccacagccgtctttctctttgcctcagccacttccttccttcgcctcaccctccccagtgcactgaagaaggtaaccgggtccagacccacgcggcgccagttctccggcgggaaggaaaaccgcgcagagaggcagcaatgaatgtggatcacgaggttaacctcttagtggaggaaattcatcgtttgggttcaaaaaatgctgatggaaagttaagcgtgaaatttggggtcctcttccgtgatgataaatgtgccaacctctttgaagcattggtaggaactcttaaagctgcaaaacgaaggaagattgtaacatatccaggagagctgcttctgcaaggtgttcatgatgatgttgacattatattactgcaagattaatgtggtttacatatctttatgtactgccattttttgtttctggtaaactggaatataaagtgaaagaacaaacatttgaacatacttaatgtatttttatagaactttgtaaacgaaaggagattcatgttttagaagtctgtccttttttatatcttgaaagaaaatctatgtatgatgctataaaataaatcctattatttttctcaggaatctggttaggaattgcaggcaatgagattttttgcggggcagggatgggaatgtttgttcataaataattagacattttctatagatatttgacattctgcgaaagcaacaagcaaactgaagaccaactcctatgagaaatattatgatgtttatgtaataaagacatgtaactgtctt

SEQ ID NO: 4 - protein, 81 aa, homo sapiens
MNVDHEVNLLVEEIHRLGSKNADGKLSVKFGVLFRDDKCANLFEALVGTLKAAKRRKIVTYPGELLLQGVHDDVDIILLQD

SEQ ID NO: 5 - DNA, 780 bp, rattus norvegicus; identical to SEQ ID NO: 1

SEQ ID NO: 6 - DNA, 835 bp, homo sapiens; identical to SEQ ID NO: 3

SEQ ID NO: 7 - DNA, 28 bp, rattus norvegicus
tcgcttctcactttgacggagtgtgaag

SEQ ID NO: 8 - DNA, 28 bp, rattus norvegicus
tgtcaagtgtggcagagactaagaatgg